(19) United States
(12) Patent Application Publication
(10) Pub. No.: US 2026/0041375 A1
(43) Pub. Date: Feb. 12, 2026

(54) NON-INVASIVE BIOSIGNAL MEASUREMENT DEVICE AND METHOD BASED ON SKIN EFFECT REMOVAL

(71) Applicant: Korea I. T. S. Co., Ltd., Seoul (KR)

(72) Inventors: Ki Doo KIM, Seoul (KR); Tae Ho KWON, Goyang-si (KR); M SHIFAT HOSSAIN, Seoul (KR)

(73) Assignee: Korea I. T. S. Co., Ltd., Seoul (KR)

(21) Appl. No.: 18/796,073

(22) Filed: Aug. 6, 2024

(51) Int. Cl. A61B 5/00; A61B 5/0205; A61B 5/024; A61B 5/145

(52) U.S. Cl. CPC A61B 5/7203; A61B 5/0205; A61B 5/14532; A61B 5/02416

(57) ABSTRACT

A non-invasive biosignal measurement device based on skin effect removal, includes: a signal measurement instrument measuring a PPG signal of a measurement subject in contact with the skin on one side of a specific body part of the measurement subject; a signal reception unit receiving the PPG signal from the signal measurement instrument; and a ratio value calculation unit removing noise due to a skin effect from the PPG signal, and then calculating ratio values at different wavelengths for measuring a biosignal including blood sugar and glycated hemoglobin of the measurement subject. The signal measurement instrument includes a first sensor module including an optical barrier and a second sensor module for removing the skin effect.

### BACKGROUND

The present disclosure relates to biosignal measurement technology, and more particularly, to a method and hardware design technology for removing noise that may be included in a PPG signal through contact between a non-invasive bio-signal measurement instrument using the PPG signals and the skin.

Diabetes is a metabolic disease characterized by hyperglycemia caused by dysfunction or secretion of insulin required for blood sugar control in the body. Chronic hyperglycemia due to diabetes causes damage and dysfunction in each organ of the body, and in particular, the chronic hyperglycemia causes microvascular complications shown in the retina, kidneys, and nerves, and macrovascular complications such as arteriosclerosis, cardiovascular, and cerebrovascular diseases, resulting in increased mortality.

However, diabetic patients can reduce the worsening or complication rate of diabetes by controlling blood sugar, losing weight, and taking medication. Therefore, diabetic patients must frequently measure their own blood sugar levels to manage their blood sugar levels and undergo regular glycated hemoglobin (HbA1C) tests, which are as important a treatment indicator as diabetic patients' blood sugar levels.

The glycated hemoglobin (HbA1c) test is a test that determines the extent to which the hemoglobin in red blood cells, which plays a role in transporting oxygen in the blood, has been glycated, and reflects a change of the blood sugar level for the past 3 to 4 months depending on the average lifespan of red blood cells. Since glucose naturally exists in normal people, hemoglobin is glycated to some extent in our blood, and the normal value varies depending on the test method, but usually up to 5.6% is normal.

In diabetic patients, as the concentration of glucose in the blood increases, the level of glycated hemoglobin also increases. Therefore, the direction of future treatment is determined by looking at these results, which clearly reveal the level of blood sugar control so far.

Meanwhile, in the conventional method of measuring glycated hemoglobin (HbA1c), a capillary blood sample is obtained by collecting blood from a vein in the arm of the measurement subject or pricking the tip of the finger with a small, pointed needle, and the concentration of glycated hemoglobin (HbA1c) is measured using the obtained blood. This invasive method of measuring glycated hemoglobin has the problem of increasing the burden of blood collection on measurement subjects and providing inaccurate values in cases where a short red blood cell lifespan, pregnancy, or kidney disease is present.

Related Art: Korean Patent No. 10-0871074 (published on Nov. 28, 2008)

### SUMMARY

In view of the above, the present disclosure provides a device and a method of non-invasive biosignal measurement based on skin effect removal, which are capable of measuring a biosignal more accurately by removing noise which may be included in a PPG signal through contact between a non-invasive biosignal measurement instrument using the PPG signal and the skin.

According to embodiments of the present disclosure, a non-invasive biosignal measurement device based on skin effect removal includes: a signal measurement instrument measuring a PPG signal of a measurement subject in contact with the skin on one side of a specific body part of the measurement subject; a signal reception unit receiving the PPG signal from the signal measurement instrument; and a ratio value calculation unit removing noise due to a skin effect from the PPG signal, and then calculating ratio values at different wavelengths for measuring a biosignal including blood sugar and glycated hemoglobin of the measurement subject.

The signal measurement instrument may include a first sensor module including an optical barrier and a second sensor module for removing the skin effect.

The first sensor module may further include a light emitting diode (LED) radiating incident light having a specific wavelength value toward one side of the body part and a photo detector (PD) detecting light derived via the skin from the incident light, and the optical barrier may be disposed between the LED and the PD, and may block noises according to intrinsic reflection and extrinsic reflection in a process in which the incident light detours the skin.

The second sensor module may be formed in a cylinder structure having a specific height based on a bottom surface which is in contact with the skin, and may include the LED and the PD disposed on the same plane on a top surface having the cylinder structure in line.

The specific body part may include a portion where capillaries existing under the skin are able to be detected, depending on the thickness of the skin.

The ratio value calculation unit may define noises due to intrinsic reflection, extrinsic reflection, and a melanin effect as the noise according to the skin effect, and generate a light reception equation related to the corresponding noises.

The ratio value calculation unit may refine the PPG signal by removing variables related to the corresponding noises from the light reception equation.

The ratio value calculation unit may define an alternative function of replacing each of the variables related to the extrinsic reflection and the melanin effect.

The ratio value calculation unit may calculate source signals applied to the alternative function using RGB skin reflection values collected through the second sensor module.

The ratio value calculation unit may construct a prediction model that receives AC and DC values for optical signals of different wavelength values collected through the first sensor module, and the RGB skin reflection value collected through the second sensor module as an input, and generates ratio values at different wavelengths as an output.

The ratio value calculation unit may construct the prediction model by using a genetic symbolic regression (GSR) model.

According to embodiments of the present disclosure, a non-invasive biosignal measurement method based on skin effect removal includes: measuring a PPG signal of a measurement subject by a signal measurement instrument which is in contact with the skin on one side of a specific body part of the measurement subject; receiving the PPG signal from the signal measurement instrument by a signal reception unit; removing noise due to a skin effect from the PPG signal by a ratio value calculation unit; and calculating ratio values at different wavelengths for measuring a biosignal including blood sugar and glycated hemoglobin of the measurement subject by the ratio value calculation unit.

The removing of the noise may include constructing a prediction model that receives AC and DC values for optical signals of different wavelength values collected through the first sensor module, and the RGB skin reflection value collected through the second sensor module as an input, and generates ratio values at different wavelengths as an output.

The removing of the noise may include constructing the prediction model by using a genetic symbolic regression (GSR) model.

The biosignal measurement method may further include: constructing a mathematical model between ratio values at different wavelengths, and biosignal values including the blood sugar and glycated hemoglobin; and calculating the biosignal of the measurement subject by using the mathematical model.

The disclosed technology may have the following effects. However, since it is not meant that a specific embodiment should include all of the following effects or merely include the following effects, the scope of the disclosed technology is not to be construed as being limited thereby.

According to an embodiment of the present disclosure, a device and a method of non-invasive biosignal measurement based on skin effect removal can measure a biosignal more accurately by removing noise which may be included in a PPG signal through contact between a non-invasive biosignal measurement instrument using the PPG signal and the skin.

### BRIEF DESCRIPTION OF THE DRAWINGS

FIG. 1 is a diagram describing a biosignal measurement system according to the present disclosure.

FIG. 2 is a diagram describing a system configuration of a biosignal measurement device of FIG. 1.

FIG. 3 is a diagram describing a functional configuration of the biosignal measurement device of FIG. 1.

FIG. 4 is a flowchart describing a non-invasive biosignal measurement method based on skin effect removal according to the present disclosure.

FIG. 5 is a diagram describing noise for a PPG signal.

FIG. 6 is a diagram illustrating an embodiment of a signal measurement instrument according to the present disclosure.

### DETAILED DESCRIPTION

A description of the present disclosure is merely an embodiment for a structural or functional description and the scope of the present disclosure should not be construed as being limited by an embodiment described in a text. That is, since the embodiment can be variously changed and have various forms, the scope of the present disclosure should be understood to include equivalents capable of realizing the technical spirit. Further, it should be understood that since a specific embodiment should include all objects or effects or include only the effect, the scope of the present disclosure is limited by the object or effect.

Meanwhile, meanings of terms described in the present application should be understood as follows.

The terms “first,” “second,” and the like are used to differentiate a certain component from other components, but the scope of should not be construed to be limited by the terms. For example, a first component may be referred to as a second component, and similarly, the second component may be referred to as the first component.

It should be understood that, when it is described that a component is “connected to” another component, the component may be directly connected to another component or a third component may be present therebetween. In contrast, it should be understood that, when it is described that an element is “directly connected to” another element, it is understood that no element is present between the element and another element. Meanwhile, other expressions describing the relationship of the components, that is, expressions such as “between” and “directly between” or “adjacent to” and “directly adjacent to” should be similarly interpreted.

It is to be understood that the singular expression encompasses a plurality of expressions unless the context clearly dictates otherwise and it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

In each step, reference numerals (e.g., a, b, c, etc.) are used for convenience of description, the reference numerals are not used to describe the order of the steps and unless otherwise stated, it may occur differently from the order specified. That is, the respective steps may be performed similarly to the specified order, performed substantially simultaneously, and performed in an opposite order.

The present disclosure can be implemented as a computer-readable code on a computer-readable recording medium and the computer-readable recording medium includes all types of recording devices for storing data that can be read by a computer system. Examples of the computer readable recording medium may include a ROM, a RAM, a CD-ROM, a magnetic tape, a floppy disk, an optical data storage device, and the like. Further, the computer readable recording media may be stored and executed as codes which may be distributed in the computer system connected through a network and read by a computer in a distribution method.

If it is not contrarily defined, all terms used herein have the same meanings as those generally understood by those skilled in the art. Terms which are defined in a generally used dictionary should be interpreted to have the same meanings as the meanings in the context of the related art, and are not interpreted as ideal meanings or excessively formal meanings unless clearly defined in the present application.

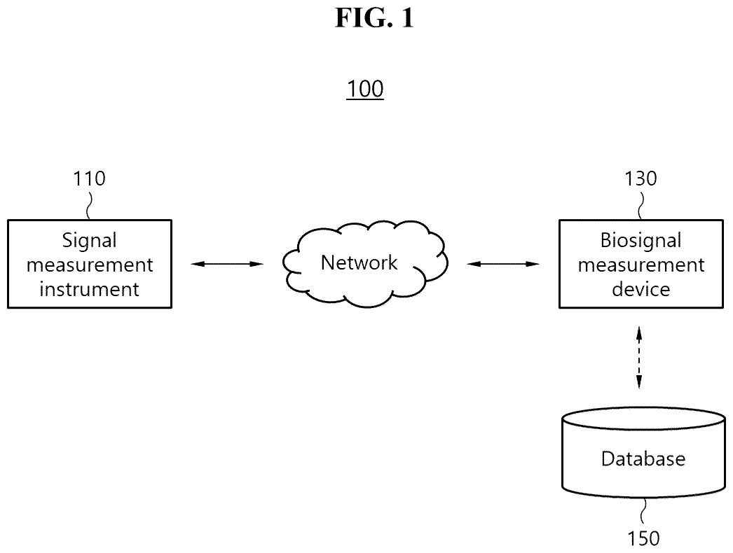

FIG. 1 is a diagram describing a biosignal measurement system according to the present disclosure.

Referring to FIG. 1, the biosignal measurement system 100 may include a signal measurement instrument 110, a biosignal measurement device 130, and a database 150.

The signal measurement instrument 110 may correspond to a computing device that measures a PPG signal while being in contact with the skin on one side of a specific body part of a measurement subject. In FIG. 1, the signal measurement instrument is illustrated as a device independent of the biosignal measurement device 130, but the signal measurement instrument 110 is not particularly limited thereto and, if necessary, may be included and implemented as a component of the biosignal measurement device 130.

Meanwhile, the specific body part of the measurement subject may include a portion where capillaries existing under the skin are able to be detected depending on the thickness of the skin. For example, the specific body part may include fingers, wrists, forehead, cheeks, ears, etc., but are not particularly limited thereto, and may include various body parts depending on an implementation form or an installation condition of the signal measurement instrument 110.

In addition, the signal measurement instrument 110 may be implemented as one device constituting the biosignal measurement system 100 according to the present disclosure, and the biosignal measurement system 100 may be transformed and implemented in various forms according to a method for solving a skin effect for non-invasive biosignal measurement and a hardware design.

In an embodiment, the signal measurement instrument 110 may be implemented as a wearable device capable of measuring the PPG signal of the measurement subject. For example, the signal measurement instrument 110 may be implemented as a smart watch, and when the PPG signal is measured through the smart watch, the signal measurement instrument 110 may transmit the PPG signal to the biosignal measurement device 130.

Further, the signal measurement instrument 110 may be implemented as various devices which are connected to the biosignal measurement device 130 and are enabled to operate. For example, the signal measurement instrument 110 may be implemented as a smartphone, a laptop, or a computer, but is, of course, not limited thereto.

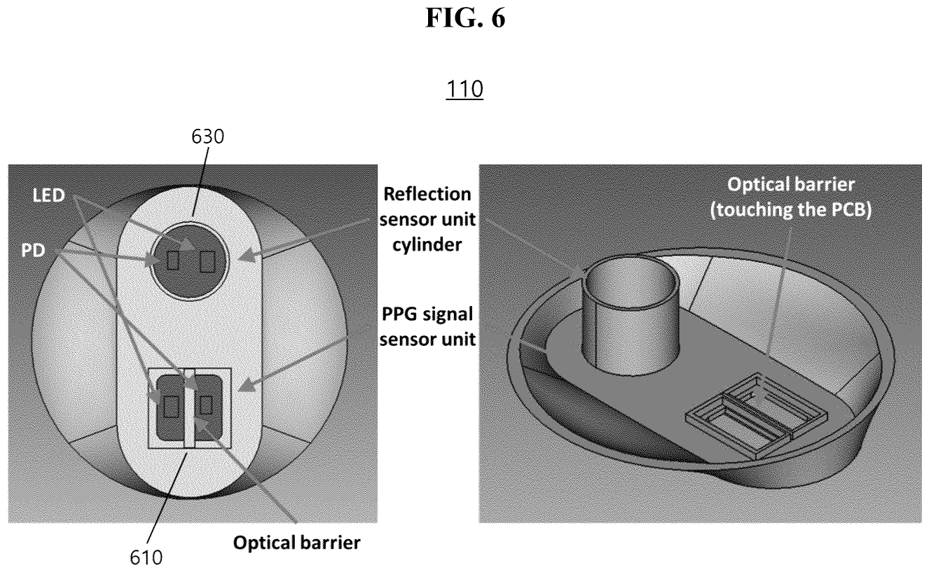

Meanwhile, the signal measurement instrument 110 may be connected to the biosignal measurement device 130, and a plurality of signal measurement instruments 110 may also be simultaneously connected to the biosignal measurement device 130. A specific configuration of the signal measurement instrument 110 will be described in more detail in FIG. 6.

The biosignal measurement device 130 may be implemented as a server corresponding to a computer or a program which performs the non-invasive biosignal measurement method based on skin effect removal according to the present disclosure. In addition, the biosignal measurement device 130 may be connected to the signal measurement instrument 110 through a wired network or a wireless network such as Bluetooth, WiFi, or LTE, and may transmit and receive data to and from the signal measurement instrument 110 through a network. Further, the biosignal measurement device 130 may be implemented to operate in connection with an independent external system (not illustrated in FIG. 1).

In an embodiment, the biosignal measurement device 130 may be implemented by being included in the signal measurement instrument 110. That is, the biosignal measurement device 130 may be implemented in a form of an independently executable module, and may be implemented by being included as a component of the signal measurement instrument 110. As a result, the signal measurement instrument 110 may perform an operation of measuring the biological signal of the measurement subject using the PPG signal and providing the result.

In an embodiment, the biosignal measurement device 130 may be implemented as a cloud server, and as a result, the signal measurement instrument 110 may transmit signal data to the biosignal measurement device 130 through the network.

The database 150 may correspond to a storage device that stores various information required during the operation of the biosignal measurement device 130. For example, the database 150 may store information about the PPG signal measured from the measurement subject or model information for skin effect removal and biosignal measurement, but is not particularly limited thereto, the biosignal measurement device 130 may store information collected or processed in various forms during the process of performing the non-invasive biosignal measurement method based on skin effect removal according to the present disclosure.

Further, in FIG. 1, the database 150 is illustrated as a device independent of the biosignal measurement device 130, but is not particularly limited thereto, and may be, of course, implemented by being included in the biosignal measurement device 130 as a logical storage device.

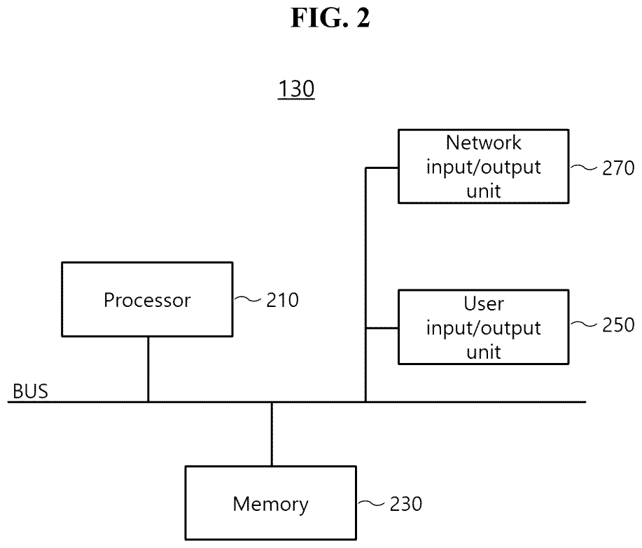

FIG. 2 is a diagram describing a system configuration of a biosignal measurement device of FIG. 1.

Referring to FIG. 2, the biosignal measurement device 130 may include a processor 210, a memory 230, a user input/output unit 250, and a network input/output unit 270.

The processor 210 may execute a non-invasive biosignal measurement procedure based on skin effect removal according to an embodiment of the present disclosure, and manage the memory 230 that is read or written in this process, and schedule a synchronization time between a volatile memory and a non-volatile memory. The processor 210 may control the overall operation of the biosignal measurement device 130 and is electrically connected to the memory 230, the user input/output unit 250, and the network input/output unit 270 to control data flow therebetween. The processor 210 may be implemented as a Central Processing Unit (CPU) or a Graphics Processing Unit (GPU) of the biosignal measurement device 130.

The memory 230 may include an auxiliary storage device implemented as the non-volatile memory such as a solid state disk (SSD) or a hard disk drive (HDD) and used to store all data required for the biosignal measurement device 130, and may include a main storage device implemented as the volatile memory such as a Random Access Memory (RAM). In addition, the memory 230 may store a set of instructions that are executed by the electrically connected processor 210 to execute the non-invasive biosignal measurement method based on skin effect removal according to the present disclosure.

The user input/output unit 250 may include an environment for receiving a user input and an environment for outputting specific information to a user, and include an input device including an adapter such as, for example, a touch pad, a touch screen, an on-screen keyboard, or a pointing device, and an output device including an adapter such as a monitor or a touch screen. In an embodiment, the user input/output unit 250 may correspond to a computing device connected through a remote connection, and in such case, the biosignal measurement device 130 may be performed as an independent server.

The network input/output unit 270 provides a communication environment for connection to the signal measurement instrument 110 through the network, and may include an adapter for communication such as, for example, Local Area Network (LAN), Metropolitan Area Network (MAN), Wide Area Network (WAN), and Value Added Network (VAN). Further, the network input/output unit 270 may be implemented to provide short-range communication functions such as WiFi and Bluetooth or wireless communication functions of 4G or higher for wireless transmission of learning data.

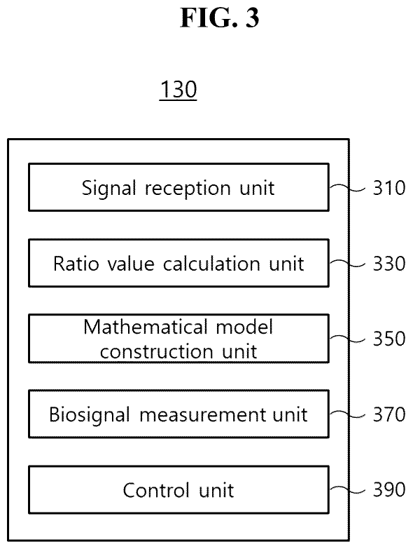

FIG. 3 is a diagram describing a functional configuration of the biosignal measurement device of FIG. 1.

Referring to FIG. 3, the biosignal measurement device 130 may include a signal reception unit 310, a ratio value calculation unit 330, a mathematical model construction unit 350, a biosignal measurement unit 370, and a control unit 390.

The signal reception unit 310 may receive the PPG signal of the measurement subject from the signal measurement instrument 110. Here, a photoplethysmogram (PPG) signal may correspond to an optically acquired plethysmogram that may be used to detect a change in blood volume that change with contraction and relaxation of the heart in the microvascular layer of the tissue. The signal reception unit 310 may operate in link with the signal measurement instrument 110, and the received PPG signal may be stored and managed through the database 150.

In an embodiment, the signal reception unit 310 may perform at least one preprocessing operation for the PPG signal. For example, the signal reception unit 310 may apply a filter to the PPG signal, remove a signal outside a predetermined range among the PPG signals, and normalize the PPG signal to a value within a specific range. The signal reception unit 310 may selectively perform various preprocessing operations depending on an operating environment of the biosignal measurement system 100.

The ratio value calculation unit 330 may receive the PPG signal from the signal measurement instrument 110, remove noise due to the skin effect from the PPG signal, and then calculate ratio values at different wavelengths for measuring a biosignal including blood sugar and glycated hemoglobin of the measurement subject. The signal measurement instrument 110 measures an optical signal reflected on the skin of the measurement subject through a PD and analyzes the intensity of the optical signal to calculate a signal value related to the PPG signal of the measurement subject. At this time, the measured value may vary depending on the skin color of the measurement subject, and the ratio value calculation unit 330 may perform an operation for removing noise signals included in the PPG signals included in the PPG signal in order to solve inaccuracy of biosignal measurement resulting from this.

In an embodiment, the ratio value calculation unit 330 may generate a light reception equation related to noise due to intrinsic reflection, extrinsic reflection, and melanin effect. The PPG signal may contain various noises due to various factors, and may include four major factors that add noise in the process of measuring the PPG signal through the signal measurement instrument 110 constituted by an LED and the PD. That is, four major factors are 1) LED spectrum and PD sensitivity, 2) internal reflection, 3) external reflection, and 4) melanin effect (i.e., skin effect due to melanin). The ratio value calculation unit 330 may define noise for each factor as a variable in the equation, and based on this, generate a light reception equation for the optical signal received through the PD of the signal measurement instrument 110.

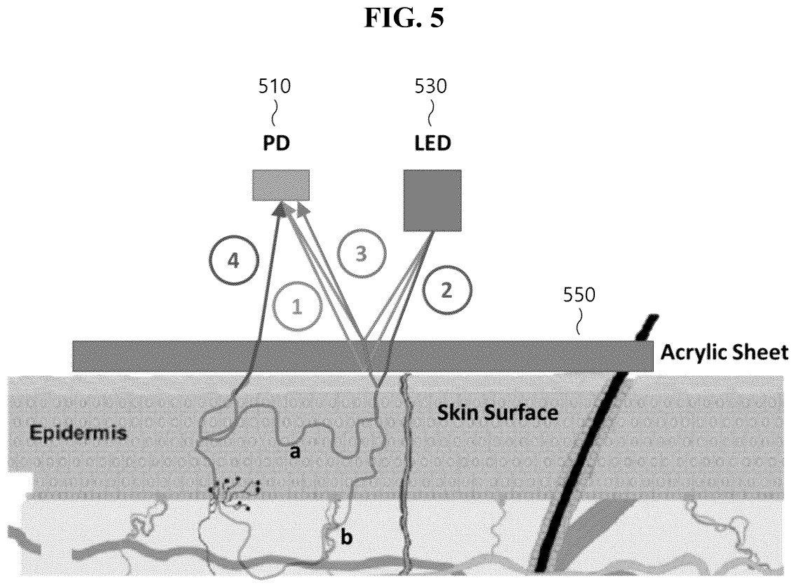

More specifically, the ratio value calculation unit 330 may define the light reception equation related to the intrinsic reflection, the extrinsic reflection, and melanin effect.

$${I}_{received} = {I}_{in} + {I}_{ex} ( λ , Mel , Bil ) + {I}_{Mel} ( α ( λ ) , {I}_{4} ) \qquad [ Equation 1 ]$$

Where I_{received} represents the intensity of received light, Iᵢₙ represents the intensity of light due to the intrinsic reflection, Iₑₓ represents the intensity of light due to the extrinsic reflection, I_{Mel} represents the intensity of light due to the melanin skin effect, λ represents a wavelength, and Mel and Bil represent skin pigmentation and bilirubin, respectively. In other words, the signal measurement instrument 110 may irradiate the skin with light and measure the light derived by being reflected or transmitted by the skin, and at this time, the intensity of the measured light may be expressed as a sum of the intensities of light due to the intrinsic reflection, the extrinsic reflection, and the melanin effect. The light reception equation will be described in more detail in FIG. 5.

In an embodiment, the ratio value calculation unit 330 may refine the PPG signal by removing variables related to corresponding noises from the light reception equation. That is, the ratio value calculation unit 330 may obtain a signal from which noise caused by various factors is removed based on the light reception equation, and measure the PPG signal based thereon. For example, variables related to noise due to the intrinsic reflection and the extrinsic reflection are removed from the light reception equation expressed as Equation 1 above, and then the intensity of light due to the melanin (i.e., skin effect) is removed from the intensity of light measured by the signal measurement instrument 110 to extract a noise-free light intensity I₄.

In an embodiment, the ratio value calculation unit 330 may define an alternative function of replacing a variable related to the melanin effect in the light reception equation. In the light reception equation expressed as Equation 1 above, the noise due to the intrinsic reflection may be generated by an acrylic sheet of the signal measurement instrument 110, and removed by an optical barrier of a first sensor module included in the signal measurement instrument 110. Accordingly, a signal by the intrinsic reflection may be easily refined in the signal measured by the signal measurement instrument 110.

Unlike this, since the signals due to the extrinsic reflection and the melanin effect are not only complex in definition but also difficult to construct a mode for each configuration, it may be very difficult to extract a noise-free signal I₄. However, the signal due to the extrinsic reflection may be easily removed through the optical barrier of the first sensor module formed in a structure in which the acrylic sheet and the PCB are physically separated. Therefore, the ratio value calculation unit 330 replaces only the variable related to the melanin effect with the alternative function to change the light reception equation to a form more suitable for extracting the noise-free signal I₄.

More specifically, the signal due to the extrinsic reflection Iₑₓ may be expressed as a first alternative function for a skin surface color and reflectivity Rₛₖᵢₙ. At this time, the skin surface color may be expressed as a second alternative function for melanin and bilirubin based on many studies on a relationship between the skin pigmentation and the skin reflectance. In other words, the signal Iₑₓ due to the extrinsic reflection may be expressed as the first or second alternative function as in Equation 2 below, and theoretically removed based on a signal measured by a second sensor module, but here, it is described that the signal Iₑₓ is physically removed through the optical barrier.

$${I}_{ex} \approx {f}_{1} ( Skin surface color , {R}_{skin} ) \approx {f}_{2} ( Melanin , Bilirubin ) \qquad [ Equation 2 ]$$

In addition, the ratio value calculation unit 330 may replace the signal according to the melanin effect with a third alternative function for the skin pigmentation (e.g., melanin, bilirubin, etc.) and the noise-free signal. That is, the signal I_{Mel} according to the melanin effect may be expressed as the third alternative function as in Equation 3 below.

$${I}_{Mel} \approx {f}_{3} ( Melanin , Bilirubin , {I}_{4} ) \qquad [ Equation 3 ]$$

In an embodiment, the ratio value calculation unit 330 may calculate source signals applied to the alternative function using RGB skin reflection values collected through the second sensor module. In the light reception equation, a similar noise source exists in a model for I_{Mel}, but simplification of the model may be impossible. As a result, the ratio value calculation unit 330 may correspond the RGB skin reflection value measured at a predetermined distance from the skin surface to a fourth alternative function for the skin pigmentation, and may be expressed as Equation 4 below.

$$( R , G , B ) \approx {f}_{4} ( Melanin , Bilirubin ) \qquad [ Equation 4 ]$$

Here, values of (R, G, B) may be measured through the second sensor module of the signal measurement instrument, and the second sensor module is implemented in a cylindrical structure and spaced apart from the skin surface at a predetermined distance (e.g., 17 mm) to generate the values of (R, G, B). That is, when the R, G, and B values are measured at a predetermined distance from the human skin surface, the R, G, and B values may be expressed as a function of the skin pigmentation (e.g., melanin, bilirubin, etc.). Since a signal received at a predetermined distance is a non-oscillating signal, the signal received at the predetermined distance may be similar to an extrinsic reflection component. Accordingly, it may be regarded that lights that penetrate the skin are largely absorbed by turbid media and then do not reach the PD.

According to Equation 4 above, signal I, a specific function corresponding to the second alternative function f₂ expressing the signal Lex due to the extrinsic reflection may be derived, and expressed as in Equation 5 below.

$${g}_{1} ( R , G , B ) \approx {f}_{2} ( Melanin , Bilirubin ) \approx {I}_{ex} \qquad [ Equation 5 ]$$

In addition, the ratio value calculation unit 330 may calculate signal values for melanin and bilirubin of the alternative function using the RGB skin reflection values collected through the second sensor module, and expressed as in Equation 6 below.

$$Melanin \approx {g}_{2} ( R , G , B ) \qquad [ Equation 6 ]$$

$$Bilirubin \approx {g}_{3} ( R , G , B )$$

As a result, the ratio value calculation unit 330 may calculate the source signals by using the RGB skin reflection value, and g₂ and g₃, and extract the noise-free signal I₄ from I_{Mel} by applying the source signal values to the light reception equation.

In an embodiment, the ratio value calculation unit 330 may construct a prediction model that receives AC and DC values for optical signals of different wavelength values collected through the first sensor module, and the RGB skin reflection value collected through the second sensor module as an input, and generates ratio values at different wavelengths as an output. The ratio value calculation unit 330 may predict a ratio value for the noise-free signal through a pre-constructed prediction model instead of directly calculating a noise-free signal component through the light reception equation based on the RGB skin reflection value measured through the signal measurement instrument 110. The prediction model may be constructed in advance by learning data collected though the signal measurement instrument 110, and the learning data may include all AC and DC values, and RGB skin reflection values for optical signals of various wavelength values.

Meanwhile, the ratio value generated as the output through the prediction model may be defined through a ratio equation defined by the intensity of the PPG signal in different wavelengths, and expressed as in Equation 7 below.

$$R = \frac{{( \frac{Δ I}{I} )}_{{λ}_{1}}}{{( \frac{Δ I}{I} )}_{{λ}_{2}}} \qquad [ Equation 7 ]$$

Where R represents the ratio equation, ΔI represents a difference value in intensity of light between a peak value of a valley value of the PPG signal, I represents the intensity of derived light, and λ₁ and λ₂ are represent different wavelength values.

In an embodiment, the ratio value calculation unit 330 may construct the prediction model by using a Genetic Symbolic Regression (GSR) model. The Genetic Symbolic Regression (GSR) model may correspond to a model created by directly modeling a function related to relationship between dependent and independent variables for data given in regression analysis. That is, the ratio value calculation unit 330 uses AC and DC values, and RGB skin reflection values for optical signals of different wavelengths as the independent variables and ratio values at different wavelengths as the dependent variables to construct the GSR model for a correlation between the variables as the prediction model. As a result, the biosignal measurement device 130 may acquire ratio values at different wavelengths that ensure validity for biosignal measurement when appropriate measurement data is given through the signal measurement instrument 110 based on a final model of GSR.

The mathematical model construction unit 350 may construct a mathematical model between ratio values at different wavelengths and biosignal values including blood sugar and glycated hemoglobin. Here, the input of the mathematical model may correspond to ratio values at different wavelengths, and the output may correspond to a biosignal value. The mathematical model construction unit 350 may learn ratio values and biosignal values collected from various users to predict biosignal values corresponding to the ratio values.

For example, the mathematical model constructed by the mathematical model construction unit 350 may be expressed as in Equation 8 below.

$${S}_{b} = f ( {R}_{1} , {R}_{2} ) \qquad [ Equation 8 ]$$

Where, S_{b} may represent the biosignal value, f may represent the mathematical model function, and R₁ and R₂ may correspond to ratio values at different wavelengths, respectively. The mathematical model constructed by the mathematical model construction unit 350 may be stored and managed in the database 150. Meanwhile, the mathematical model may also be implemented as a neural network model, a deep learning model, and an artificial intelligence model.

In an embodiment, the mathematical model construction unit 350 may calibrate the mathematical model based on actual values of biosignals such as blood sugar or glycated hemoglobin (HbA1c) measured directly from the measurement subject. The mathematical model construction unit 350 may improve the prediction accuracy of the mathematical model by correcting the mathematical model constructed in advance using actual values measured from humans. In other words, correction of the mathematical model may be performed in a direction of reducing the difference between the actual value and the predicted value.

The biological signal measurement unit 370 may calculate the biosignal of the measurement subject using the mathematical model. Here, the biosignal may include blood sugar, glycated hemoglobin (HbA1c), etc. That is, the biosignal measurement unit 370 may calculate the biosignal value by applying the ratio value calculated by the ratio value calculation unit 330 to a previously constructed mathematical model. The biosignal measurement unit 370 may output the measured biosignal values through various graphic interfaces. The biosignal measurement unit 370 may store and manage measured biosignal values for each user, and selectively provide measurement information for a specific period or changes over time.

The control unit 390 may control an overall operation of the biosignal measurement device 130, and manage a control flow or a data flow among the signal reception unit 310, the ratio value calculation unit 330, the mathematical model construction unit 350, and the biosignal measurement unit 370.

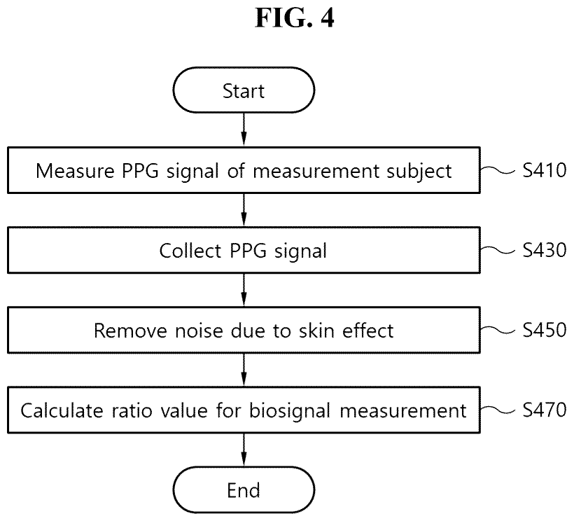

FIG. 4 is a flowchart describing a non-invasive biosignal measurement method based on skin effect removal according to the present disclosure.

Referring to FIG. 4, the biosignal measurement device 130 can measure the PPG signal of the measurement subject through the signal measurement instrument 110 (step S410). The biosignal measurement device 130 may receive the PPG signal measured by the signal measurement instrument 110 through the signal reception unit 310 (step S430).

Further, the biosignal measurement device 130 removes noise due to a skin effect from the PPG signal through the ratio value calculation unit 330 (step 450), and calculate ratio values at different wavelengths for measuring a biosignal including blood sugar and glycated hemoglobin of the measurement subject (step S470).

FIG. 5 is a diagram describing noise for a PPG signal.

Referring to FIG. 5, the signal measurement instrument 110 may radiate incident light having a specific wavelength value toward the skin surface through the LED 530 while being in contact with the skin surface. Thereafter, the signal measurement instrument 110 may receive derived light received by being reflected by the skin surface or transmitted through the skin through the PD 510.

In FIG. 5, signals \textcircled{1} and \textcircled{3} may correspond to signals resulting from intrinsic reflection and extrinsic reflection, respectively. Since there is no partition between the LED 530 and the PD 510 and an acrylic sheet is used, an intrinsic reflection component Iᵢₙ may affect the signal received through PD 510.

The transmitted signal \textcircled{2} may interact with the skin surface and cause two phenomena. The first phenomenon may be direct reflection from the surface as in \textcircled{3}, and may correspond to the extrinsic reflection component Iₑₓ in the signal received through the PD 510. Signal \textcircled{4} may correspond to an expected part of derived light that passes through the skin and interacts with blood and non-blood components according to a theoretical model.

Meanwhile, since signal \textcircled{3} depends on skin reflection Rₛₖᵢₙ, it may not be easy to separate signal \textcircled{3} like intrinsic reflection \textcircled{1}, and signal \textcircled{3} may correspond to a function related to wavelength and ethnicity (more specifically, melanin (Mel) and bilirubin (Bil)). Signal \textcircled{3} as the extrinsic reflection component may be expressed as Iₑₓ(λ, Mel, Bil).

Further, signal \textcircled{4} may correspond to a skin effect signal including the noise-free signal, and may be expressed as I_{Mel}(α(λ),I₄).

The bio-signal measurement device 130 according to the present disclosure may define light reception equations for signals \textcircled{1}, \textcircled{2}, and \textcircled{4}, and remove noise (melanin effect) according to the skin effect from the PPG signal measured by the signal measurement instrument 110 by using the corresponding the light reception equation.

FIG. 6 is a diagram illustrating an embodiment of a signal measurement instrument according to the present disclosure.

Referring to FIG. 6, the biosignal measurement device 130 can measure the PPG signal of the measurement subject through the signal measurement instrument 110. At this time, the signal measurement instrument 110 may correspond to a device that measures the PPG signal while being in contact with the skin on one side of a specific body part of the measurement subject.

Further, the signal measurement instrument 110 may be implemented to include a sensor module composed of an LED that radiates light of a specific wavelength and a PD that detects the intensity of the light. More specifically, the signal measurement instrument 110 may be implemented to include a first sensor module 610 and a second sensor module 630.

Here, the first sensor module 610 may be implemented to include an LED that irradiates one side of the body part with incident light with a specific wavelength value, a photo detector that detects light derived from the incident light via the skin, and an optical barrier disposed between the LED and the PD, and blocking noises according to intrinsic reflection and extrinsic reflection generated in a process in which the incident light between the LED and the PD detours the skin.

Further, the second sensor module 630 may be implemented to be formed in a cylinder structure having a specific height based on a bottom surface which is in contact with the skin, and to include the LED and the PD disposed on the same plane on a top surface having the cylinder structure in line.

In the case of FIG. 6, the signal measurement instrument 110 may correspond to a watch type signal measurement instrument 110 worn on a wrist of the measurement subject. A body forming an exterior of the signal measurement instrument 110 may be divided into an upper body part and a lower body part, and specifically, FIG. 6 illustrates an embodiment of a lower body member.

That is, the lower body member may be formed in a structure in which the first and second sensor modules are disposed on the same plane. In an embodiment, the first sensor module 610 may be implemented to be square shaped, and the second sensor module 630 may be implemented to be rounded. In particular, the first sensor module 610 may be implemented to include the optical barrier that blocks between the LED and the PD, and the optical barrier may block light directly projected or reflected from the LED to the PD.

In addition, the first sensor module may be implemented to include an acrylic sheet on the side in contact with the skin surface, and in the case of the first sensor module 610, the acrylic sheet may also be separated by the optical barrier. The optical barrier may separate an internal space of the first sensor module 610 as well as the PPG PCB coupled to the lower body member. Meanwhile, the acrylic sheet may be coupled to prevent skin damage such as burns due to heat when being in contact with the skin. The second sensor module 630 may be implemented in the cylinder structure, and the bottom surface having the cylinder structure which is in contact with the skin surface need not be coupled to the acrylic sheet. The top surface having the cylinder structure may not be coupled to the acrylic sheet, and only the LED and the PD are coupled to reduce internal reflection in the cylinder structure.

In an embodiment, the second sensor module 630 may also be implemented to selectively include the optical barrier separating the LED and the PD. In another embodiment, distances between the LED and the PD included in the first and second sensor modules, respectively, may be set to 3 mm or more or 5 mm or less, but are not particularly limited thereto.

The present disclosure has been described with reference to the preferred embodiments of the present disclosure, but those skilled in the art will understand that the present disclosure can be variously modified and changed without departing from the spirit and the scope of the present disclosure which are defined in the appended claims.

[Detailed Description of Main Elements]

| | |
|---|---|
| 100: Biosignal measurement system | |
| 110: Signal measurement instrument | |
| 130: Biosignal measurement device | |
| 150: Database | |
| 210: Processor | 230: Memory |
| 250: User input/output unit | 270: Network input/output unit |
| 310: Signal reception unit | 330: Ratio value calculation unit |
| 350: Mathematical model construction unit | 370: Biosignal measurement unit |
| 390: Control unit | |

1. A non-invasive biosignal measurement device based on skin effect removal, comprising:
a signal measurement instrument measuring a PPG signal of a measurement subject in contact with the skin on one side of a specific body part of the measurement subject;
a signal reception unit receiving the PPG signal from the signal measurement instrument; and
a ratio value calculation unit removing noise due to a skin effect from the PPG signal, and then calculating ratio values at different wavelengths for measuring a biosignal including blood sugar and glycated hemoglobin of the measurement subject,
wherein the signal measurement instrument includes a first sensor module including an optical barrier and a second sensor module for removing the skin effect.

2. The non-invasive biosignal measurement device based on skin effect removal of claim 1, wherein the first sensor module further includes
a light emitting diode (LED) irradiating one side of the body part with incident light having a specific wavelength value and a photo detector (PD) detecting the light derived via the skin from the incident light, and
the optical barrier is disposed between the LED and the PD, and blocks noises according to intrinsic reflection and extrinsic reflection in a process in which the incident light detours the skin.

3. The non-invasive biosignal measurement device based on skin effect removal of claim 1, wherein the second sensor module is formed in a cylinder structure having a specific height based on a bottom surface which is in contact with the skin, and includes the LED and the PD disposed on the same plane on a top surface having the cylinder structure in line.

4. The non-invasive biosignal measurement device based on skin effect removal of claim 1, wherein the specific body part includes a portion where capillaries existing under the skin are able to be detected, depending on the thickness of the skin.

5. The non-invasive biosignal measurement device based on skin effect removal of claim 1, wherein the ratio value calculation unit defines noises due to intrinsic reflection, extrinsic reflection, and a melanin effect as the noise according to the skin effect, and generates a light reception equation related to the corresponding noises.

6. The non-invasive biosignal measurement device based on skin effect removal of claim 5, wherein the ratio value calculation unit removes variables related to the noises due to the intrinsic reflection and the extrinsic reflection from the light reception equation, and then corresponds the light reception equation to the PPG signal, and removes the noise due to the skin effect based on the corresponding light reception equation to refine the PPG signal.

7. The non-invasive biosignal measurement device based on skin effect removal of claim 6, wherein the ratio value calculation unit defines an alternative function of replacing the variable for the melanin effect in the corresponding light reception equation.

8. The non-invasive biosignal measurement device based on skin effect removal of claim 7, wherein the ratio value calculation unit calculates source signals applied to the alternative function using RGB skin reflection values collected through the second sensor module.

9. The non-invasive biosignal measurement device based on skin effect removal of claim 1, wherein the ratio value calculation unit constructs a prediction model that receives AC and DC values for optical signals of different wavelength values collected through the first sensor module, and the RGB skin reflection value collected through the second sensor module as an input, and generates ratio values at different wavelengths as an output.

10. The non-invasive biosignal measurement device based on skin effect removal of claim 9, wherein the ratio value calculation unit constructs the prediction model by using a genetic symbolic regression (GSR) model.

11. A non-invasive biosignal measurement method based on skin effect removal, comprising:
measuring a PPG signal of a measurement subject by a signal measurement instrument which is in contact with the skin on one side of a specific body part of the measurement subject;
receiving the PPG signal from the signal measurement instrument by a signal reception unit;
removing noise due to a skin effect from the PPG signal by a ratio value calculation unit; and
calculating ratio values at different wavelengths for measuring a biosignal including blood sugar and glycated hemoglobin of the measurement subject by the ratio value calculation unit,
wherein the signal measurement instrument includes a first sensor module including an optical barrier and a second sensor module for removing the skin effect.

12. The non-invasive biosignal measurement method based on skin effect removal of claim 11, wherein the first sensor module further includes
a light emitting diode (LED) irradiating one side of the body part with incident light having a specific wavelength value and a photo detector (PD) detecting the light derived via the skin from the incident light, and
the optical barrier is disposed between the LED and the PD, and blocks direct movement of the incident light between the LED and the PD.

13. The non-invasive biosignal measurement method based on skin effect removal of claim 11, wherein the second sensor module is formed in a cylinder structure having a specific height based on a bottom surface which is in contact with the skin, and includes the LED and the PD disposed on the same plane on a top surface having the cylinder structure in line.

14. The non-invasive biosignal measurement method based on skin effect removal of claim 11, wherein the removing of the noise includes constructing a prediction model that receives AC and DC values for optical signals of different wavelength values collected through the first sensor module, and the RGB skin reflection values collected through the second sensor module as an input, and generates ratio values at different wavelengths as an output.

15. The non-invasive biosignal measurement method based on skin effect removal of claim 14, wherein the removing of the noise includes constructing the prediction model by using a genetic symbolic regression (GSR) model.

16. The non-invasive biosignal measurement method based on skin effect removal of claim 11, further comprising:
constructing a mathematical model between ratio values at different wavelengths, and biosignal values including the blood sugar and glycated hemoglobin; and
calculating the biosignal of the measurement subject by using the mathematical model.
